(19) United States
(12) Patent Application Publication
(10) Pub. No.: US 2011/0245851 A1
(43) Pub. Date: Oct. 6, 2011

(54) REMOVABLE MEDICAL DEVICE HAVING AT LEAST ONE PATCH MEMBER

(76) Inventors: Richard W. Ducharme, Winston-Salem, NC (US); Michelle D. Martinez, Winston-Salem, NC (US); Vihar C. Surti, Winston-Salem, NC (US); Tyler E. McLawhorn, Winston-Salem, NC (US)

(21) Appl. No.: 13/074,109

(22) Filed: Mar. 29, 2011

Related U.S. Application Data
(60) Provisional application No. 61/320,137, filed on Apr. 1, 2010.

(51) Int. Cl. A61B 17/00

(52) U.S. Cl. 606/151

(57) ABSTRACT

The present embodiments provide a removable medical device, comprising a sleeve having proximal and distal ends and a lumen extending therebetween. At least one support member having an expanded deployed configuration is coupled to the sleeve using at least one permanent connector. A patch member comprising a resorbable material is coupled to at least a portion of an outer surface of the sleeve using at least one temporary connector. In use, when the support member is in the expanded deployed configuration, at least a portion of the patch member contacts the target site and fluid flows through the lumen of the sleeve. In a separate procedure, a retrieval member is operable to facilitate removal of the sleeve and support member while leaving the patch member disposed within the bodily passageway.

### PRIORITY CLAIM

This invention claims the benefit of priority of U.S. Provisional Application Ser. No. 61/320,137, entitled “Removable Medical Device Having At Least One Patch Member,” filed Apr. 1, 2010, the disclosure of which is hereby incorporated by reference in its entirety.

### BACKGROUND

The present embodiments relate generally to medical devices, and more particularly, to a removable medical device.

There are various instances in which it might be desirable or necessary to remodel a segment of a patient's tissue. As one example, it might be necessary to facilitate closure of a perforation in a bodily wall that was formed intentionally or unintentionally. An intentional perforation may be formed, for example, during surgical procedures such as translumenal procedures.

In a translumenal procedure, one or more instruments may be inserted through a visceral wall, such as the esophageal wall. For example, it may be desirable to endoscopically retrieve a lymph node situated within the mediastinal cavity, or gain access through an opening in the esophagus to perform therapies or diagnostics in the thoracic cavity.

During a translumenal procedure, a closure instrument may be used to close the perforation in the visceral wall. Various closure devices include suturing devices, t-anchors, clips, and other devices that may apply compressive forces. Depending on the structure comprising the perforation, it may be difficult to adequately close the perforation and prevent leakage of bodily fluids.

With regard to the esophagus in particular, certain closure devices that apply compressive forces may not be desirable as they may impact the structure of the passageway. Further, such devices may leave strictures from scarring that may cause complications. Moreover, it may be difficult to deploy various closure devices or perform suturing in the esophagus. Further, even if the above techniques adequately treat the target tissue, e.g., by ensuring closure of an opening without leakage, such techniques may not promote remodeling of tissue over time, and in certain instances, it may not be desirable to permanently leave certain components within the passageway.

### SUMMARY

The present embodiments provide a removable medical device, comprising a sleeve having proximal and distal ends and a lumen extending therebetween. At least one support member having an expanded deployed configuration is coupled to the sleeve using at least one permanent connector. A patch member comprising a resorbable material is coupled to at least a portion of an outer surface of the sleeve using at least one temporary connector. In use, when the support member is in the expanded deployed configuration, at least a portion of the patch member contacts the target site and fluid flows through the lumen of the sleeve. In a separate procedure, a retrieval member is operable to facilitate removal of the sleeve and support member while leaving the patch member disposed within the bodily passageway.

In one embodiment, the medical device further comprises a fixation member having proximal and distal ends, and further having an expanded deployed configuration in which the fixation member engages an inner wall of the bodily passageway. The proximal end of the fixation member is coupled to the retrieval member, and the distal end of the fixation member is coupled to the proximal end of the sleeve. At least one barb may be coupled to the fixation member and faces in a distal direction, wherein proximal retraction of the retrieval member causes the barb to disengage from the inner wall of the bodily passageway.

In one example, the patch member comprises small intestinal submucosa that is left in the passageway to promote tissue ingrowth. The patch member may cover only a selected portion of the outer surface of the sleeve. Further, the patch member may be coupled to the outer surface of the sleeve using the temporary connector to enable removal of the sleeve relative to the patch member.

Advantageously, the patch member promotes site-appropriate tissue remodeling at the target site. There are therefore no long-term forces imposed upon the target site. Further, since the sleeve and support members are retrieved, they need not be left within the patient's body.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a side view of a first embodiment of a removable medical device having a patch member.

FIG. 2 is a side view of the removable medical device of FIG. 1 with inner components shown in dashed lines, and with the proximal barrier of FIG. 1 omitted to depict a fixation member and a retrieval member.

FIG. 3 is a schematic view illustrating an esophagus and mediastinal cavity.

FIG. 4 is a side sectional view of a portion of the esophagus with a perforation formed therein.

FIG. 5 is a schematic view illustrating the medical device of FIGS. 1-2 deployed in the esophagus.

FIG. 6-7 are schematic views depicting removal of the medical device of FIGS. 1-2.

FIG. 8 is a side sectional view of a portion of the esophagus after removal of the medical device of FIGS. 1-2.

### DETAILED DESCRIPTION OF THE PREFERRED EMBODIMENTS

In the present application, the term “proximal” refers to a direction that is generally towards a physician during a medical procedure, while the term “distal” refers to a direction that is generally towards a target site within a patent's anatomy during a medical procedure.

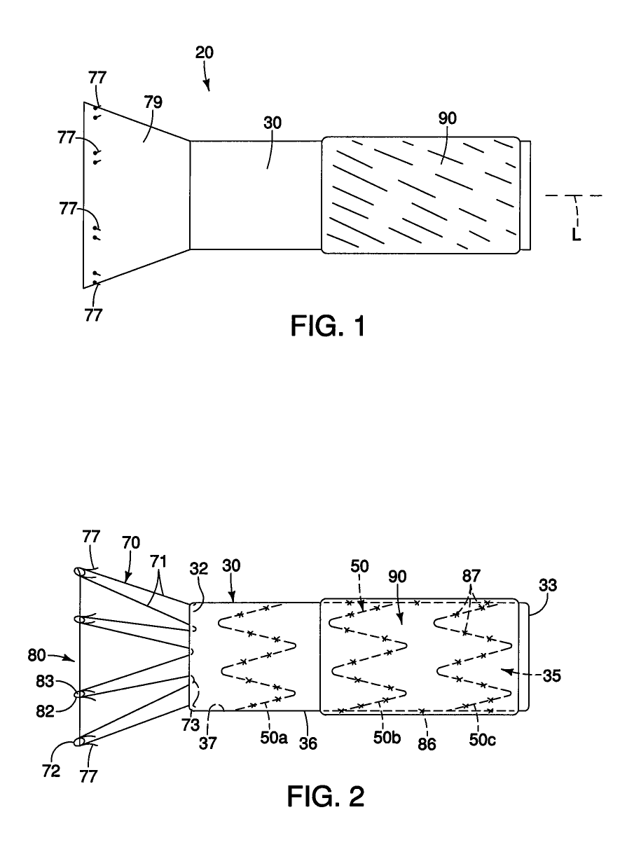

Referring to FIGS. 1-2, a first embodiment of a removable medical device 20 is shown. The medical device 20 comprises a sleeve 30 having proximal and distal ends 32 and 33, and a lumen 35 extending therebetween. The sleeve 30 further comprises outer and inner surfaces 36 and 37, respectively, as best seen in FIG. 2.

In one embodiment, the proximal end 32 of the sleeve 30 may terminate adjacent to a distal end 73 of a fixation member 70, as shown in FIG. 2. In this embodiment, the distal end 73 of the fixation member 70 may be sutured to the proximal end 32 of the sleeve 30, and the remaining portion of the fixation member 70 may be covered by a barrier 79, as depicted in FIG. 1, or remain uncovered, as depicted in FIG. 2. If the barrier 79 is used, it may be separate from, or formed integral with, the proximal end 32 of the sleeve 30.

The sleeve 30 preferably is formed from a material that is substantially impermeable to acids, food and the like. By way of example and without limitation, the sleeve 30 may be formed from urethane or polyethylene. By providing a substantially impermeable material, the sleeve 30 may effectively isolate a patch member 90 that provides a smart or “site-appropriate” tissue remodeling during the healing process, as explained in further detail below.

The medical device 20 further comprises at least one support member 50, which is coupled to the sleeve 30. In a presently preferred embodiment, the support member 50 is coupled to the inner surface 37 of the sleeve 30 using at least one permanent connector 87, as depicted in FIG. 2. As used herein, the term permanent is intended to mean that the material used is not subject to the degrative effects of acid and enzymes, or the general environment found within the esophagus or other bodily passageway, over an extended period of time, therefore allowing the device to remain intact for the intended life of the device. In one example, a plurality of permanent connectors 87 are provided in the form of non-resorbable sutures. Further, plastics may be selected, including selected polyesters, polyurethanes, polyethylenes, polyamides, silicone, or other possible materials.

In alternative embodiments, the support member 50 may be coupled to the outer surface 36 of the sleeve 30, or alternatively, disposed within layers of the sleeve 30, for example, using lamination or other known techniques.

In the embodiment of FIG. 1, three support members 50a-50c are shown, each generally comprising a zig-zag shape formed, for example, using a wire comprising a plurality of substantially straight segments having a plurality of bent segments disposed therebetween. Optionally, the plurality of support members 50a-50c may be coupled together, for example, using one or more struts extending axially along a longitudinal axis L of the medical device 20.

It will be appreciated that while three support members 50a-50c are shown, greater or fewer support members may be used. Moreover, while the support members shown comprise zig-zag configurations, the support members may alternatively comprise any number of shapes. For example, the support member 50 may comprise a pattern of interconnected struts, including diamond or other shapes as generally known in the art. The support members may be made from a woven wire structure, a laser-cut cannula, individual interconnected rings, or any other type of stent structure that is known in the art.

Moreover, the support members 50 may be made from numerous metals and alloys. The support members 50 may be made from other metals and alloys that are biased, such that they may be restrained by a delivery device prior to deployment, but are inclined to return to their relaxed, expanded configuration shown in FIG. 2 upon deployment. In a preferred embodiment, the support members 50 comprise a self-expanding nitinol or stainless steel stent. Alternatively, the support members 50 may comprise other materials such as cobalt-chrome alloys, amorphous metals, tantalum, platinum, gold and titanium. The support members 50 also may be made from non-metallic materials, such as thermoplastics and other polymers.

The medical device 20 has a reduced diameter delivery state in which it may be advanced to a target location within a vessel, duct or other anatomical site. The medical device 20 further has an expanded deployed state in which it may be configured to apply a radially outward force upon the vessel, duct or other target location, e.g., to maintain patency within a passageway. In the expanded state, fluid flow is allowed through the lumen 35 of the sleeve 30, as explained further in FIG. 5 below.

Further, as noted above, the medical device 20 may comprise a fixation member 70, which in one embodiment may comprise a series of zig-zag segments 71, each having a proximal end 72 and a distal end 73. The proximal ends 72 of the segments 71 be flared in a radially-outward direction, e.g., to engage a healthy portion of a bodily passageway. The distal ends 73 of the segments 71 may be coupled to the proximal end 32 of the sleeve 30, for example, using sutures.

The proximal ends 72 that engage the vessel wall may comprise one or more barbs 77, which preferably are oriented with their tips facing in a distal direction, as shown in FIGS. 1-2. The barbs 77 may be formed integrally with the segments 71, for example, by laser cutting. Alternatively, the barbs 77 may be externally formed and then attached to the segments 71, e.g., using an adhesive or mechanical means.

As noted above, the fixation member 70 may be covered by the barrier 79, as depicted in FIG. 1, or remain uncovered, as depicted in FIG. 2. If the barrier 79 is used, the barbs 77 may pierce through the barrier 79, as shown in FIG. 1, in order to engage an inner surface of a bodily passageway, as explained further in FIG. 5 below.

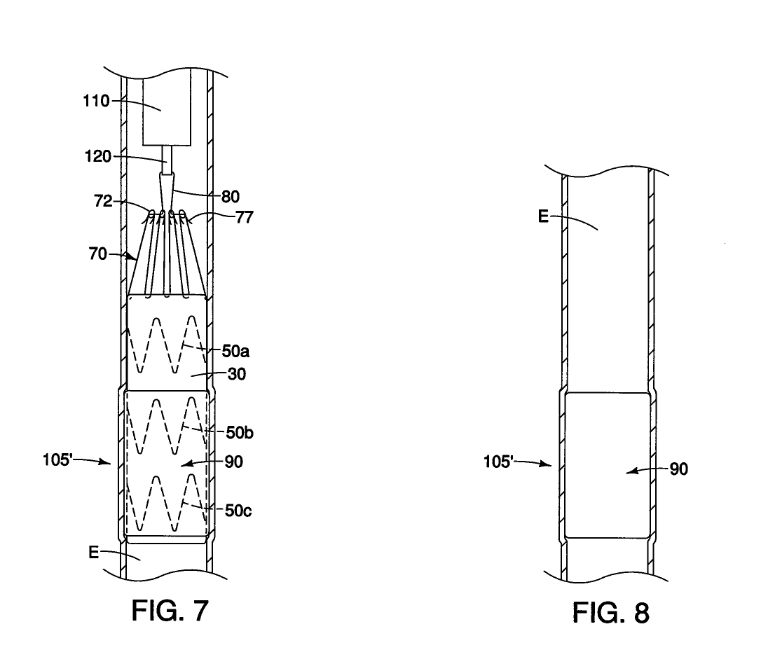

A retrieval member 80 may be coupled to the fixation member 70. In one embodiment, the retrieval member 80 comprises a loop of suture, as depicted in FIG. 2. The proximal ends 72 of the segments 71 may comprise rings 82 having bores 83 formed therein, through which the loop of suture 80 is threaded. In one example, the rings 82 may be soldered to the segments 71 near the proximal ends 72, such that portions of the rings 82 are partially co-extensive with the proximal apices of the segments 71. In use, the retrieval member 80 may be actuated to at least partially radially collapse the segments 71, thereby facilitating subsequent withdrawal of the medical device 20, as explained further in FIGS. 6-7 below.

Referring still to FIGS. 1-2, the medical device 20 further has a patch member 90, which may be disposed to surround at least a portion of the outer surface 36 of the sleeve 30. In the example shown in FIGS. 1-2, the patch member 90 covers only a distal portion of the outer surface 36 of the sleeve 30. Alternatively, the patch member 90 may cover all, or substantially all, of the outer surface 36 of the sleeve 30. In a still further alternative, multiple patch member segments may be applied to align with selected regions along the length and circumference of the sleeve 30, as needed to achieve a desired biological effect on a bodily passageway.

The patch member 90 preferably comprises a resorbable material. Solely by way of example, and without limitation, the patch member 90 may provide a smart or “site-appropriate” tissue remodeling through its three-dimensional extracellular matrix (ECM) that is colonized by host tissue cells and blood vessels, and provides a scaffold for connective and epithelial tissue growth and differentiation along with the ECM components. In one example of such a patch member that provides a site-appropriate tissue remodeling, the patch member 90 may comprise porcine small intestinal submucosa (SIS), such as SURGISIS® BIODESIGN™ Soft Tissue Graft, available from Cook Medical, Inc., Bloomington, Ind.

Preferably, the patch member 90 would be a one to four layer lyophilized soft tissue graft made from any number of tissue engineered products. Reconstituted or naturally-derived collagenous materials can be used, and such materials that are at least bioresorbable will provide an advantage, with materials that are bioremodelable and promote cellular invasion and ingrowth providing particular advantage. Suitable bioremodelable materials can be provided by collagenous ECMs possessing biotropic properties, including in certain forms angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa. The patch member 90 may also comprise a composite of a biomaterial and a biodegradeable polymer. Additional details may be found in U.S. Pat. No. 6,206,931 to Cook et al., the disclosure of which is incorporated herein by reference in its entirety.

The patch member 90 may be coupled to at least a portion of the outer surface 36 of the sleeve 30. In a presently preferred embodiment, the patch member 90 is coupled to the outer surface 36 of the sleeve 30 using at least one temporary connector 86, as depicted in FIG. 2. As used herein, the term temporary is intended to mean that the connection between the patch member 90 and the sleeve 30 is not permanent over an intended time period. For example, the temporary connector may comprise a biologically resorbable, degradable, dissolvable or erodible material. After the particular process of resorption, degradation, dissolution and/or erosion has been completed or substantially completed, the connection between the patch member 90 and the sleeve 30 is weakened or eliminated. In one example, a plurality of temporary connectors 86 are provided in the form of resorbable sutures 86, as depicted in FIG. 2. As noted above, the patch member 90 may be disposed to surround only one or more selected portions of the outer surface 36 of the sleeve 30, for example, those portions that are expected to contact a portion of a passageway for which tissue remodeling is desired.

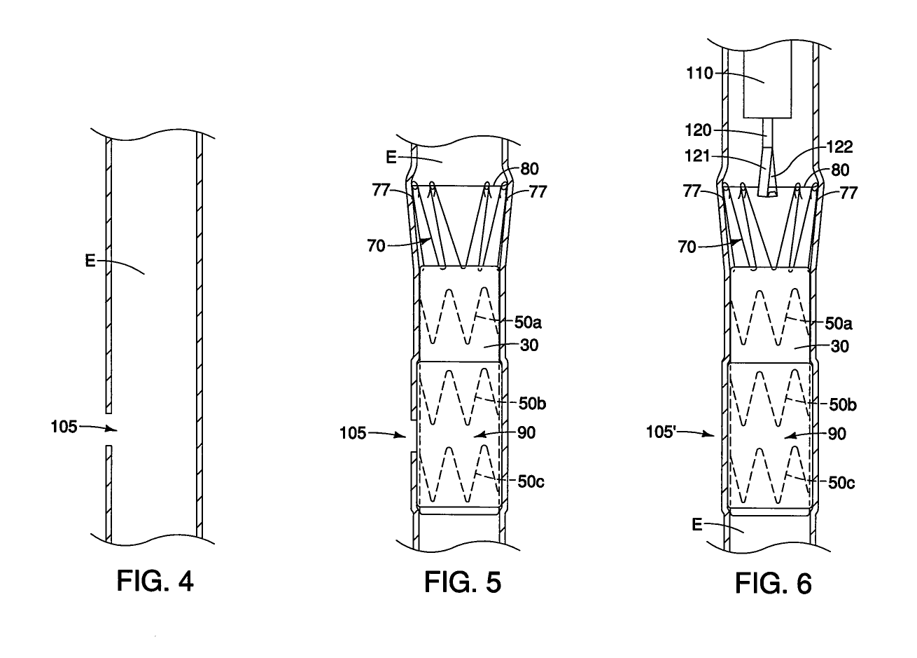

In one example, described further in FIGS. 4-8 below, the patch member 90 may promote site-appropriate tissue remodeling to facilitate closure of one or more perforations 105. Over a period of time, a portion of the medical device 20 including the sleeve 30, the support members 50 and the fixation member 70 may be retrieved from the patient's body passageway, leaving the resorbable patch member 90 within the passageway, as explained in further detail below.

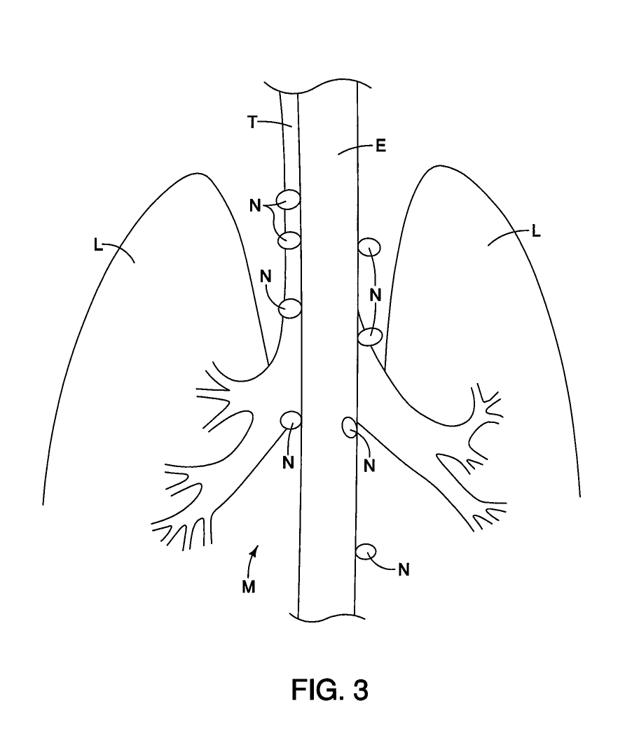

Referring now to FIG. 3, a portion of an esophagus E is shown, along with a patient's trachea T, lungs L and multiple mediastinal lymph nodes N. In a medical procedure, it may become necessary or desirable to create a perforation 105 in the esophagus E, as shown in FIG. 4. For example, in one procedure, it may be desirable to endoscopically retrieve a lymph node N situated within the mediastinal cavity, such as a malignant node. In other procedures, it may be desirable to gain access through the perforation 105 in the esophagus E to perform therapies or diagnostics in the thoracic cavity using a translumenal approach.

Referring to FIG. 5, after the perforation 105 in the esophagus E of FIG. 4 has been created, the medical device 20 of FIGS. 1-2 may be deployed. As shown in FIG. 5, after deployment the medical device 20 is positioned within the esophagus E such that the patch member 90 is aligned with the perforation 105 or otherwise contacts a target site. At this time, the support member 50 is in the expanded deployed configuration such that fluid flows through the lumen 35 of the sleeve 30. Notably, the fixation member 70 with barbs 77 is aligned with a healthy portion of the esophagus E situated proximal to the perforation 105, such that the barbs 77 engage an inner surface of the esophagus E. When fluid flows in a proximal to distal direction in the esophagus E, the barbs 77 anchor into the esophagus E to secure the medical device 20 in place, as shown in FIG. 5.

It should be noted that the medical device 20 may be deployed in the esophagus E using a suitable stent deployment system. One exemplary systems is shown in U.S. Published Application No. 2009/0281610 A1 (“the '610 publication”), which is incorporated by reference in its entirety. While the '610 publication describes one system for delivering and deploying the medical device 20 described herein, other suitable delivery and deployment systems may be used to deliver the medical device 20 in the esophagus E in accordance with the techniques described herein.

After deployment of the medical device 20 in the esophagus E as shown in FIG. 5, over time the patch member 90 promotes site-appropriate tissue remodeling to facilitate closure of the perforation 105, resulting in a remodeled tissue segment 105′. Then, in a separate procedure shown in FIGS. 6-7, the retrieval member 80 is operable to facilitate removal of the sleeve 30, the support member 50 and the fixation member 70, while leaving the patch member 90 engaged with remodeled tissue within the bodily passageway.

In a presently preferred removal technique, a commercially available esophageal overtube may be placed in the patient's esophagus E at a location proximal to the medical device 20. The esophageal overtube is not shown in FIGS. 6-7 for illustrative purposes. An end-viewing endoscope 110 may be advanced through the esophageal overtube to a position proximal to the fixation member 70, as shown in FIG. 6. A retrieval device 120, such as a rat-tooth forceps having first and second opposing members 121 and 122, may be advanced through a working lumen of the endoscope 110. Under endoscopic imaging guidance, the first and second opposing members 121 and 122 may be positioned to surround a portion of the retrieval member 80, as depicted in FIG. 6.

Referring to FIG. 7, in a next step, retrieval device 120 may be actuated, e.g., by moving the first and second opposing members 121 and 122 towards one another, to thereby engage the retrieval member 80. The retrieval device 120 then may be proximally retracted, thereby tensioning and reducing the diameter of the loop of suture 80 disposed through the rings 82 of the segments 71 of the fixation member 70. At this time, the proximal ends 72 of the segments 71 will be pulled radially inward to a reduced diameter, as shown in FIG. 7, and notably the barbs 77 will no longer be engaged with the inner surface of the esophagus E.

In a next step, the endoscope 110 and the retrieval device 120 may be proximally retracted at the same time. Since the at least one temporary connector 86 coupling the sleeve 30 to the patch member 90 is weakened or eliminated over time, as explained above, proximal retraction of the fixation member 70 achieves withdrawal of the sleeve 30 and the attached support members 50, while leaving the patch member 90 within the bodily passageway, as shown in FIG. 8. Notably, the esophageal overtube is left in place until the procedure is completed to ensure a safe removal of the components while the endoscope 110 and the medical device 20 are retracted proximally and removed from the patient.

Advantageously, the patch member 90 promotes site-appropriate tissue remodeling to facilitate closure of the perforation 105, and further remodeling of the esophageal tissue over time. There are therefore no long-term forces imposed upon the esophageal wall, and the inner diameter and structure of the esophagus is not impacted. Further, since the sleeve 30, support members 50 and fixation member 70 are retrieved, they need not be left within the patient's body.

It should be noted that while the exemplary embodiments herein depict treatment of a perforation formed in the esophagus, the medical device 20 and methods described herein may be used to treat any particular defect or condition in any vessel, duct, or other passageway.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

We claim:

1. A medical device, comprising:
a sleeve having proximal and distal ends and a lumen extending therebetween;
at least one support member coupled to the sleeve, the support member having an expanded deployed configuration, wherein the support member is permanently coupled to the sleeve using at least one permanent connector; and
a patch member coupled to at least a portion of an outer surface of the sleeve, the patch member comprising a resorbable material, wherein the patch member is temporarily coupled to the sleeve using at least one temporary connector;
wherein, when the support member is in the expanded deployed configuration, at least a portion of the patch member contacts a target site and fluid flows through the lumen of the sleeve.

2. The medical device of claim 1 wherein the temporary connector comprises at least one resorbable suture that enables removal of the sleeve relative to the patch member.

3. The medical device of claim 1 wherein the temporary connector comprises at least one degradable material that enables removal of the sleeve relative to the patch member.

4. The medical device of claim 1 wherein the permanent connector comprises at least one non-resorbable suture.

5. The medical device of claim 1 further comprising a fixation member having proximal and distal ends, and further having an expanded deployed configuration in which the fixation member engages an inner surface of the bodily passageway, wherein the proximal end of the fixation member is coupled to a retrieval member, and the distal end of the fixation member is coupled to the proximal end of the sleeve.

6. The medical device of claim 5 further comprising at least one barb coupled to the fixation member and facing in a distal direction, wherein proximal retraction of the retrieval member causes the barb to disengage from the inner surface of the bodily passageway.

7. The medical device of claim 5, wherein the fixation member comprises a series of zig-zag shaped segments, each having proximal and distal ends, and wherein the retrieval member comprises a loop of suture coupled to the proximal ends of the zig-zag shaped segments.

8. The medical device of claim 7 wherein the proximal ends of the zig-zag segments comprise rings having bores formed therein, wherein the loop of suture is threaded through the bores.

9. The medical device of claim 1 wherein the at least one support member is coupled to an inner surface of the sleeve.

10. The medical device of claim 1 wherein the patch member comprises small intestinal submucosa.

11. The medical device of claim 1 wherein the patch member covers only a selected portion of the outer surface of the sleeve.

12. A method for treating a medical condition in a bodily passageway, the method comprising:
providing a medical device comprising a sleeve having proximal and distal ends and a lumen extending therebetween, at least one support member coupled to the sleeve, and a patch member coupled to at least a portion of an outer surface of the sleeve, the patch member comprising a resorbable material;
deploying the medical device at a target site in the bodily passageway wherein, when the support member is in an expanded deployed configuration, at least a portion of the patch member contacts a target site and fluid flows through the lumen of the sleeve; and
subsequently, in a separate procedure, removing the sleeve and the support member from the bodily passageway, while leaving the patch member within the bodily passageway.

13. The method of claim 12 wherein the support member is permanently coupled to the sleeve using at least one permanent connector, and wherein the patch member is coupled to at least a portion of an outer surface of the sleeve using at least one temporary connector.

14. The method of claim 12 further comprising covering only a selected portion of the outer surface of the sleeve with the patch member.

15. The method of claim 12 further comprising:
providing a fixation member having proximal and distal ends, and further having an expanded deployed configuration in which the fixation member engages an inner surface of the bodily passageway, wherein the proximal end of the fixation member is coupled to a retrieval member, and the distal end of the fixation member is coupled to the proximal end of the sleeve, wherein the fixation member comprises a series of zig-zag shaped segments, each having proximal and distal ends, and wherein the retrieval member comprises a loop of suture coupled to the proximal ends of the zig-zag shaped segments; and
actuating the retrieval member using a grasping device,
wherein proximal retraction of the grasping device reduces a diameter of the loop of suture, thereby reducing an expanded diameter of at least a portion of the fixation member.

16. A medical device, comprising:
a sleeve having proximal and distal ends and a lumen extending therebetween;
at least one support member coupled to the sleeve, the support member having an expanded deployed configuration;
a patch member coupled to at least a portion of the outer surface of the sleeve, the patch member comprising a resorbable material;
a fixation member having proximal and distal ends, wherein the proximal end of the fixation member is coupled to a retrieval member, and the distal end of the fixation member is coupled to the proximal end of the sleeve; and
a barrier disposed proximal to the sleeve and encircling the fixation member and the retrieval member,
wherein, when the support member is in the expanded deployed configuration, at least a portion of the patch member contacts the target site and fluid flows through the lumen of the sleeve, and
wherein, in a separate procedure, the retrieval member is operable to facilitate removal of the sleeve and support member while leaving the patch member disposed within the bodily passageway.

17. The medical device of claim 16 wherein the support member is permanently coupled to the sleeve using at least one permanent connector, and wherein the patch member is coupled to at least a portion of an outer surface of the sleeve using at least one temporary connector.

18. The medical device of claim 17 wherein the permanent connector comprises at least one non-resorbable suture.

19. The medical device of claim 17 wherein the temporary connector comprises at least one resorbable suture that enables removal of the sleeve relative to the patch member.

20. The medical device of claim 16, wherein the fixation member comprises a series of zig-zag shaped segments, each having proximal and distal ends, and wherein the retrieval member comprises a loop of suture coupled to the distal ends of the zig-zag shaped segments.
